(19) United States
(12) Patent Application Publication
(10) Pub. No.: US 2006/0216774 A1
(43) Pub. Date: Sep. 28, 2006

(54) Method

(75) Inventors: Tanjore SoundaRajan Balganesh, Bangalore (IN); Santanu Datta, Bangalore (IN); Indira Ghosh, Bangalore (IN)

Correspondence Address: ASTRAZENECA R&D BOSTON, 35 GATEHOUSE DRIVE, WALTHAM, MA, 02451-1215, US

(73) Assignee: AstraZeneca AB, Sodertaije (SE)

(21) Appl. No.: 10/550,647

(22) PCT Filed: Mar. 23, 2004
(86) PCT No.: PCT/GB04/01272

§ 371 Date: Sep. 27, 2005

(30) Foreign Application Priority Data
Mar. 29, 2003 (GB) 03073293

(51) Int. Cl. C12Q 1/48; C12Q 1/26; C12Q 1/18

(52) U.S. Cl. 435/15; 435/25; 435/32

(57) ABSTRACT

A method for attenuating a microorganism which comprises inhibiting in the microorganism a metabolic pathway essential for viability, by promoting use of the substrate of the pathway in a different metabolic pathway which is non-essential to the microorganism whereby the substrate is unavailable to the essential pathway and the micro organism is attenuated.

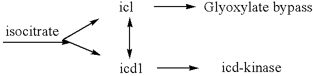

The present invention relates to novel methods for targeting intracellular pathogens. In particular it relates to methods of inhibiting metabolic pathways essential for the viability of a microorganism.

In the presence of glucose during aerobic respiration all organisms generate ATP with a concomitant release of two CO₂ molecules through the TCA/Krebs cycle. Microorganisms in addition have the unique ability to generate ATP through a shunt of the TCA cycle called the glyoxylate pathway. This pathway is essential for the organism when grown in C2 carbon source like acetate. In absence of the glyoxylate bypass both the carbons of acetate would be converted to CO₂ and there would not remain any residual carbon to be utilized for the synthesis of cellular constituents. The glyoxylate bypass consists of enzymes isocitrate lyase (icl) and malate synthase(ms). In E. coli the bypass enzymes are induced by acetate.

The branch point between the glyoxylate bypass and the TCA/Krebs cycle in E. coli is a well-studied area documenting the utility of compensatory phosphorylation. In E. coli the genes icl and ms are on an operon along with the kinase aceK. The major control of flux at this branch point is through a reversible phosphorylation of the branch point enzyme isocitrate dehydrogenase (icd1). The two branch point enzymes isocitrate lyase (icl) and isocitrate dehydrogenase (icd1) compete for the common substrate isocitrate. In the presence of glucose the large differential of Km (icl/icd1˜80) and the relatively low concentration of isocitrate (30 μM) compared to the high Km of icl (604 μM) makes all the isocitrate flow through icd1. However in presence of two carbon substrates like acetate not only does the isocitrate concentration increase to 111 μM, more importantly a specific kinase; isocitrate dehydrogenase kinase (acek) is induced which phosphorylates icd1 thereby inactivating the enzyme. Under this condition a significant amount of isocitrate can flow through the glyoxylate bypass thus preventing the loss of the carbon atoms as CO₂. The details of this control at the branch point of a metabolic pathway where the flow of substrate is ultra sensitive to factors such as the substrate concentration, the differential Km and the reversible inactivation of the branch point enzyme through phosphorylation by a specific kinase has been thoroughly worked out by Koshland and co-workers. (LaPorte, Walsh & Koshland J Biol. Chem. 1984 vol259,14068-14075; Walsh & Koshland J Biol. Chem. 1985, vol 260, 8430-8437).

Recently the role of the glyoxylate shunt has become the subject of intensive investigation because of the landmark work where it was shown by McKinney and co-workers (McKinney et al., Nature, 2000 vol. 406 735-738) that tuberculosis strains in which the icl gene have been inactivated were eliminated from activated macrophages at a significantly higher rate than the parent strain with the wild type icl gene. Similarly the icl mutant had reduced virulence in immunocompetent animals. In contrast the icl mutant grew in in-vitro conditions as well as the wild type strain. These findings established the fact that the icl gene product isocitrate lysase and thus the glyoxylate pathway was essential for intracellular survival. In M. tuberculosis there are two icl genes for encoding isocitrate lyase the significance of which is not understood. Sacchettini et al., 2002 WO 02/33118 A2) describe and claim isocitrate lyase as the target for anti-tubercular drug development.

The genes encoding the glyoxylate shunt enzymes have also been shown to regulate pathogenicity and persistence in other intracellular pathogens. icl mutants of C. albicans (Lorenz et al, Nature, 2001 vol 412, 83-86,) are unable to multiply within macrophages and malate synthase mutants of the plant pathogen Rhodococcus fascians fail to persist in the leafy gall disease caused by the wild type (Vereecke et al Trends in Microbiology, 2002, vol 10, 485-488). Taken together the data support the involvement of the “glyoxylate shunt” in the intracellular survival of bacterial and plant pathogens. This may be a reflection of the type of substrates available within the environmental niche.

The present invention is based on the discovery that it is possible to inhibit a metabolic pathway essential for the viability of a microorganism by diverting the substrate of the pathway into a different metabolic pathway. A particular advantage is that it is not necessary to inhibit one or more enzymes in the essential pathway.

We call this unique mode of inactivating an enzyme/pathway “substrate diversion mechanism”. It represents a novel mechanism of action in drug discovery.

Therefore in a first aspect of the invention we provide a method for attenuating a microorganism, which comprises inhibiting, in the microorganism a metabolic pathway essential for viability by promoting use of the substrate of the pathway in a different metabolic pathway which is non-essential to the microorganism whereby the substrate is unavailable to the essential pathway and the microorganism is attenuated.

By “metabolic pathway essential for viability” we mean a metabolic pathway that must operate for the microorganism to exist in a state other than cell death or leading to cell death. This includes cell growth, resting and latent states.

By “substrate of the pathway” we mean the only or main substrate of the pathway such that the viability of the organism is dependent on the availability of this.

By “attenuating a microorganism” we mean killing it or at least decreasing its growth rate to an extent where the immune system of a human or animal host is able to contain or eliminate the microorganism. or a state, for example in a plant host, in which the microbe is rendered incapable of causing disease. This may mean that metabolism via the essential metabolic pathway is either fully or partially inhibited. It may not be necessary to fully inhibit the pathway to achieve useful pharmacological effects.

In a further aspect of the invention the essentiality of the metabolic pathways is determined by the presence or absence of particular nutrients. By this we mean that the microorganism may be adapted to remain viable in the absence of a particular nutrient such as glucose, glycerol or amino acids or nucleotides (in case of specific auxotrophs) by means of compensatory or shunt pathways which become essential in the absence of such nutrient and the normal pathway for metabolising such nutrient thus becomes the non-essential pathway.

The method of the invention may be used with any convenient microorganism, these include pathogens which have a restricted environment as far as nutrient options are concerned like Candida albicans (fungal infection), Leptosphaeria maculans (fungal plant pathogen, Idnurm etal, Eukaryotic Cell 2002, vol 1. 719-724) & Rhodococcus fascians (plant pathogen) This is strikingly exemplified in many pathogens whose pathogenicity is dependent on intracellular survival and multiplication, a stage closely associated with the persistence or latency stage of Mycobacterium tuberculosis. The list of organisms which encompass such stage is given in Table 1 hereinafter.

The person of ordinary skill will be able to select convenient essential and non-essential metabolic pathways and substrates for use in the method of the invention. By way of non-limiting example it is convenient to target metabolic branch points. At such points enzymes from the two metabolic pathways compete for the common substrate. Control at branch points may depend on a number of factors including the growth medium, substrate concentration, the differential Km and the reversible inactivation of enzymes beyond the branch point.

It is known that a branch point in a metabolic pathway provides a unique mode of control of substrates through a pathway. One of the first studies on branch point effect and disease was done on gout and regulation of purine biosynthesis (Hershfield & Seegmiller, Horiz Biochem Biophys. 1976; vol 2:134-62.

Arachidonic acid is the branch point substrate between two families of potent physiological effectors, one including cyclooxygenase which leads to the synthesis of prostaglandins and thromboxanes, the other involving lipoxygenase which leads to the synthesis of leukotrines and lipoxins. While aspirin and other NSAID are available as potent inhibitors of cyclooxygenase no effective inhibitor of lipoxygenase are available (Prigge et al Biochimie 1997, vol 79, 629-636).

The target for anticancer therapy IMP dehydrogenase (IMDH) is at the metabolic branch point of purine nucleotide synthesis pathway. Blockage of the conversion of IMP to XMP by IMPDH inhibitors lead to a depletion of the guanylate (GMP, GDP, GTP and dGTP pools) leading to the death of the rapidly dividing cancer cells (Franchetti & Grifantini, Curr. Med.Chem. 1999, vol6, 599-614). Computer simulation of simple biochemical pathways containing divergent branch has been used to study the interaction of two inhibitors that straddle the branch point (Jackson, Cancer Res. 1993 vol. 53, 3998-4003). However to the best of our knowledge the concept of substrate diversion for inhibition of a branched pathway has not been anticipated.

Promoting use of the substrate in the non-essential metabolic pathway may be achieved by restoring function to one or more enzymes in the pathway. In a typical branch point where one branch leads to an essential pathway while the other branches to a non-essential one, the essential pathway may be activated by diverting the substrate through the non essential path using the control parameters mentioned earlier. This strategy may also be used for pathways having more than two branches. Some of the metabolic pathways which have branch points are the Glyoxylate pathway, Glycolysis, Pentose Phosphate pathway, Purine Salvage pathway and Phenylalanine and Tyrosine metabolism.

A particular essential metabolic pathway is the glyoxylate pathway in the TCA/Krebs cycle. As previously mentioned microorganisms have the unique ability to generate ATP through the glyoxylate pathway.

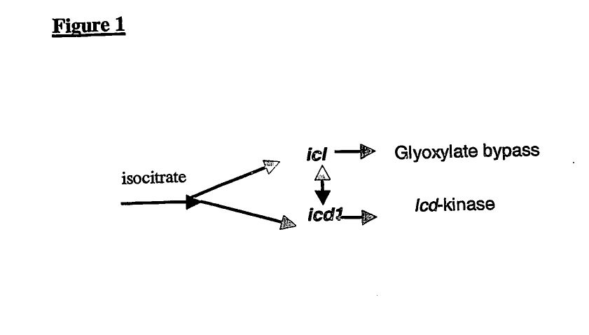

The flow of the substrate isocitrate is generally through the TCA cycle when the organism grows in glucose. However as mentioned in the prior art there is a partial diversion through the glyoxylate bypass when the carbon source is acetate (C2 carbon source). This is outlined in FIG. 1. This regulation which depends on the Km isocitrate for icl and icd1, its velocity and the phosphorylation of icd1 by aceK has been elaborately worked out by Koshland and co-workers (LaPorte, Walsh & Koshland J Biol. Chem. 1984 vol259,14068-14075; Walsh & Koshland J Biol. Chem. 1985, vol 260, 8430-8437). It has also been shown that acek mutant strain of E. coli grows well like the wild type strain when the carbon source is C6 like glucose. In contrast it is starved to death under C2 carbon source (LaPorte, Thorsness & Koshland J. Biol. Chem 1985, vol26, 10563-10568). This indicates the essentiality of substrate diversion.

By way of non-limiting example the glyoxylate pathway has been found in the folllowing organisms:

E. coli, S. typhi, S. typhimurium, Y. pestis, S. flexneri, X. fastidosa, X. canipestris, X. axonopodis, V. cholerae, V. vulnifucus, P. aeroginosa, P. putida, S. onedensis, R. solanacearum, M. loti, S. meliloti, A. tumefaciens, B. meliloti, B. suis, B. japonicum, C. crescentus, O. iheyensis, M. tuberculosis, M. leprae, C. glutanicum, C. efficiens, S. coelicolor, D. Radiodurans, S. solfataricus, C. elegans, A. thzaliana, S. cervisiae

For the glyoxylate shunt to operate analogous to that observed in E. coli, the trio of genes isocitrate dehydrogenase (icd1), isocitrate lyase (icl) and isocitrate dehydrogenase kinase (icdk) should be present. Though icd1 and icl have been annotated in a large number of organisms icdk has only been identified in a few. The list given below in Table 1 names the organisms where the trio of the genes have been identified. These genes (with its id) were identified from the KEGG database. (www.genome.adjp/kegg).

TABLE 1

| Organism | Icd1 | icl | icdk |
|---|---|---|---|
| E. coli | b1136 | b4015 | b4016 |
| E. coliJ | JW1122 | JW3975 | JW3976 |
| E. coli0157 | Z1865 | Z5601 | Z5602 |
| E. coli0157J | Ecs1608 | Ecs4933 | ECs4934 |
| E. coliCFT073 | c1517 | c4972 | c4974 |
| S. typhi | STY1278 | STY4402 | STY4403 |
| S. typhimurium | STM1238 | STM4184 | STM4185 |
| Y. pestis | YPO1641 | YPO3725 | YPO3724 |
| Y. pestisKIM | y1802 | y0016 | y0018 |
| S. flexneri | SF1155 | SF4081 | SF4082 |
| X. campestris | XCC0967 | XCC0238 | XCC3780 |
| X. axonopodis | XAC1046/3835 | XAC0257 | XAC3832 |
| P. aeruginosa | PA2623 | PA2634 | PA1376 |
| P. putida | PP4012 | PP4116 | PP4011/4565 |
| R. solanacearum | RSO1106 | RSO4640 | RSO3245 |

In a particular aspect of the invention the microorganism is Mycobacterium tuberculosis. As mentioned earlier the glyoxylate bypass of this bacteria has been proposed to be essential for its intracellular survival within the macrophage. In order to target the glyoxylate bypass of M. tuberculosis by the mechanism of substrate diversion as mentioned earlier it is essential to identify the target enzyme responsible for the diversion, the M. tuberculosis equivalent of icd-kinase. This enzyme is a unique target for inhibiting through the method of the invention as cells inhibited of this enzyme would not be able to survive within the macrophage. As this kinase has not previously been identified in M. tuberculosis, we describe below in the Examples the approach we have employed to identify and characterise this enzyme as pknG.

In a further aspect of the invention we disclose a method for identifying compounds that attenuate Mycobacterium tuberculosis which method comprises testing compounds in a test system for their ability to bind to pknG and prevent autophosphorylation of pknG.

In a further aspect of the invention we disclose a method for identifying compounds that attenuate Mycobacterium tuberculosis which method comprises testing compounds in a test system for their ability to bind to pknG and prevent phosphorylation of icd1.

In a further aspect of the invention we disclose a method for identifying compounds that attenuate Mycobacterium tuberculosis which method comprises testing compounds in a test system for their ability to bind to icd1 and prevent phosphorylation of icd1 by pkzG.

In a further aspect of the invention we disclose a method for identifying compounds that attenuate Mycobacterium tuberculosis which method comprises testing compounds in a test system for their ability to prevent the phosphorylation and or inactivation of icd1.

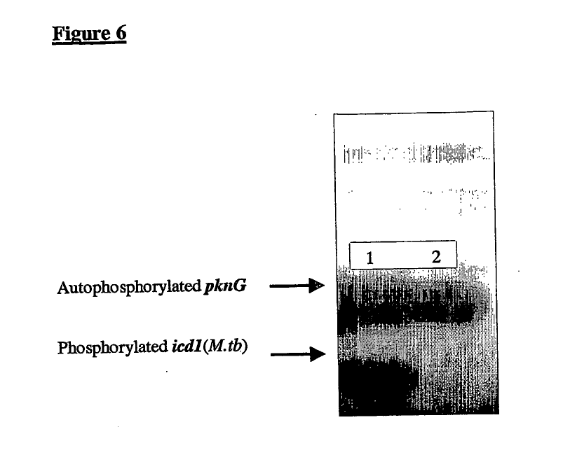

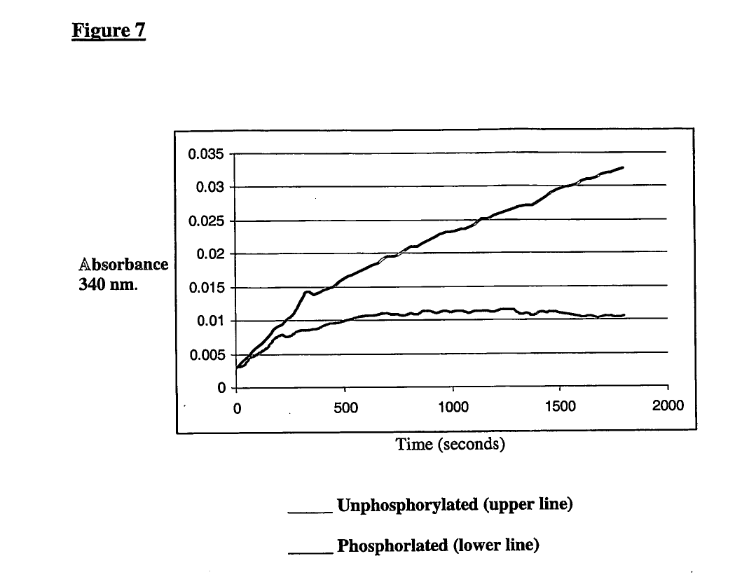

Convenient test systems for use in the above methods will be apparent to the scientist of ordinary skill. By way of non-limiting example such test systems will conveniently comprise as appropriate either the pknG enzyme or the icd1 enzyme or both enzymes, the enzyme may be coupled to a reporter system capable of indicating as appropriate the presence or absence of autophosphorylation of pknG or phosphorylation of icd1. Since the phosphorylation of icd1 will lead to its concomitant inactivation, the degree of phosphorylation can be indirectly measured by monitoring its activity in 340 n.m. which measures the formation of NADPH FIG. 7). Using radiolabelled (32γ) ATP we can monitor the phopshorylation of pknG and/or icd1 (FIG. 6). Also the degree of phophorylation of either pknG and icd1 can be directly measured in a standard ELISA format by using either generic phophoserine antibodies or antibodies specific to either of the enzymes Alternately, other methods available to detect phosphorylation of proteins or peptides, e.g. the IMAP technology that uses fluorescence anisotropy may be used. Instead of the entire icd1 protein, peptide/s representing fragments of icd1 that is phosphorylated by pknG can be used to monitor the phosphorylation, provided pknG is able to phosphorylate the said peptide.

In a further aspect of the invention we provide an antimicrobial compound identified by one of the above method aspects of the invention.

The invention will now be illustrated but not limited by reference to the following non-limiting Examples and Figures wherein:

FIG. 1 illustrates the common isocitrate substrate for the icd1 enzyme in the TCA cycle and the icl enzyme in the glyoxylate bypass. icd-kinase inactivates icd1 and thus turns on the glyoxylate bypass.

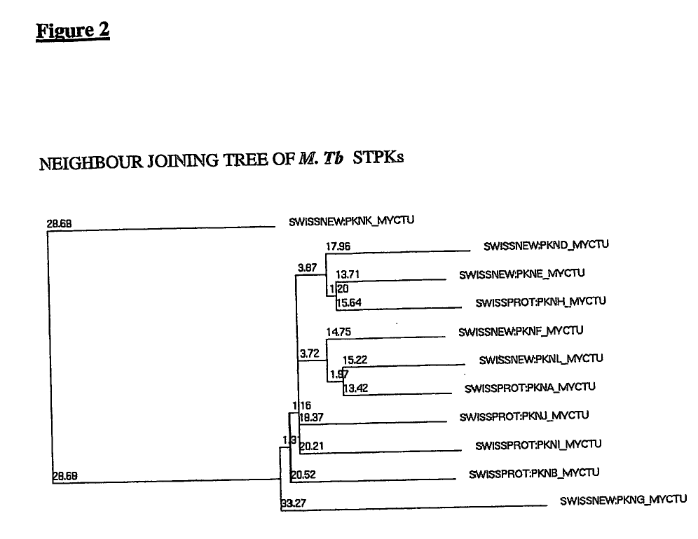

FIG. 2 shows a neighbour joining tree for the 11 M. tuberculosis Ser-Thr protein kinases.

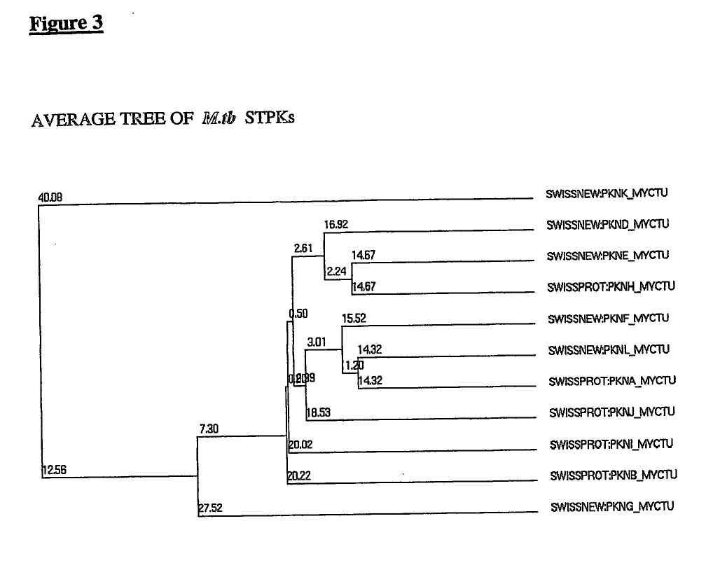

FIG. 3 shows an average tree for the 11 M. tuberculosis Ser-Thr protein kinases.

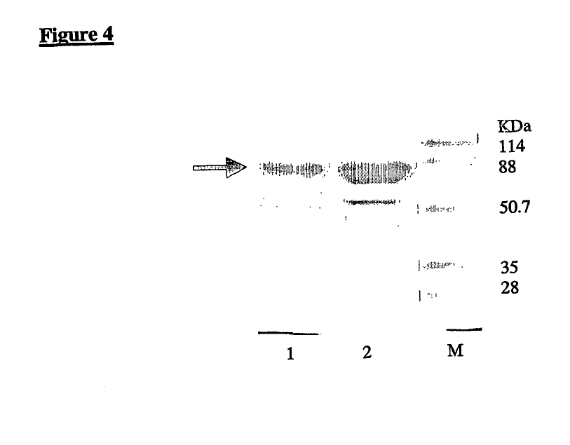

FIG. 4 illustrates the purification of pknG. Lane 1 and 2 indicate the purified pknG (indicated by the arrow). Lane M indicates the molecular weight markers.

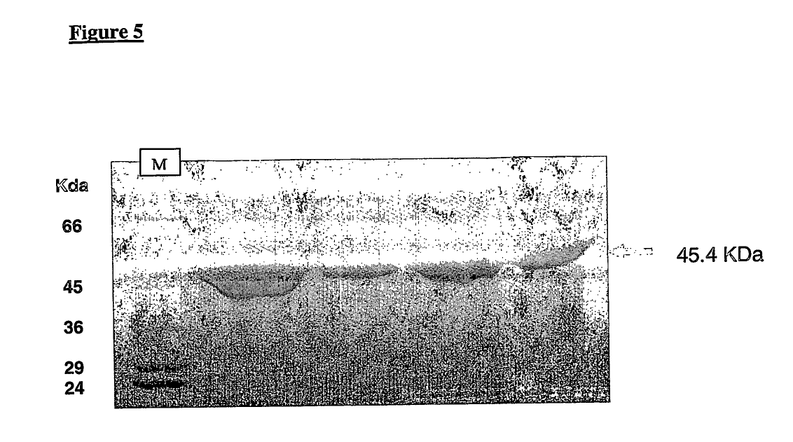

FIG. 5 illustrates the purification of isocitrate dehydrogenase. The purified protein was monitored by 10% SDS-PAGE and is shown as a 45.4 Kda band.

FIG. 6 shows the results of experiments to show that pknG specifically phosphorylates icd1. The results show that pknG specifically phosphorylates icd1 (lane 1) whereas when E. coli icd is added as a substrate, only autophosphorylation of pknG occurs (lane 2).

FIG. 7 shows a graph of the typical reaction of icd1 in the presence and absence of pknG. The y-axis shows absorbance as measured at 340 nm and the x-axis shows time in seconds.

### EXAMPLE 1

Protein Kinases in M. tuberculosis; Identification of pknG as the icd-Kinase in M. Tuberculosis

The protein kinase ‘signature’, including all 11 domains that are conserved according to Hanks (Hanks, S. K., A. M. Quinn, and T. Hunter. Science, 1988 vol. 241: 42-51) is present in all the eleven M. tuberculosis Ser-Thr protein kinases (STPK) that have been annotated. An additional gene, pknM, which was previously annotated as a putative STPK-encoding gene, does not possess any recognizable kinase signatures. The homology between pknM and the carboxy-terminal region of pknH probably explains the original annotation. Alignment of the STPK family members revealed that 15 residues are absolutely conserved across the group. All kinases other than pknI possess a lysine in the active site [Hanks domain VIb (DXKPXN, where X is any amino acid)], which is characteristic of STPKs. pknI has an asparagine at this position, which is unusual for an STPK; however, the other conserved residues in the domain indicate that pknI is a Ser/Thr kinase.

In order to identify the putative icd-kinase in M. tuberculosis a search strategy based on the following assumption was adopted.
1. The sequence alignment of all the eleven STPK of M. tuberculosis was compared with the icd-kinase of E. coli (aceK).
2. Since the icd-kinase activity occurs in conjunction with all the TCA cycle enzymes, it should occur in the cytosol, the enzyme should lack transmembrane domains.
3. The specific icd-kinase of M. tuberculosis should have least homology with the other STPKs.
4. It is presumed that a homolog of the putative icd-kinase in M. tuberculosis is also present in M. leprae, as it is also a intracellular pathogen.
   Homology with aceK

A bioinformatics approach was taken to identify the putative homologous kinase among the annotated M.tb STPKs with aceK of E. coli. The effort was not restricted within the sequence analogy or motif searching but rather took advantage of the known relationship between the STPK's of M.tb and the structural homology displayed between aceK and eukaryotic STPKs. The main features identified were the followings;

The strongly and weakly homologous residues differ among the 12 sequences. Based on the strongly homologous residues K and L in the second conserved motif and the weakly homologous residue A in the third conserved motif, it is seen that pknG, pknL, pknA and pknB are most similar to E. coli aceK.

TABLE 2
Transmembrane Domains(TM) in M. tuberculosis STPKs.

| Gene | Rv No: | TM domain |
|---|---|---|
| PknA | 0015c | + |
| PknB | 0014c | + |
| PknD | 0931c | + |
| PknE | 1743c | + |
| PknF | 1746c | + |
| PknG | 0410c | |
| PknH | 1266c | + |
| PknI | 2914c | + |
| PknJ | 2088c | + |
| PknK | 3080c | |
| PknL | 2176c | + |

It is seen from Table 2 that the TM domain is absent in the genes pknG and pknK indicating that probably these two STPKs act in the cytosolic milieu.

Homology and Clustering of the STPKs

TABLE 3
PERCENTAGE ID MATRIX

| | PKNB | PKND | PKNE | PKNF | PKNG | PKNH | PKNI | PKNJ | PKNK | PKNL |
|---|---|---|---|---|---|---|---|---|---|---|
| PKNA | 31.8 | 31.72 | 31.11 | 27.88 | 21.65 | 31.34 | 26.7 | 30.58 | 26.94 | 35.18 |
| PKNB | | 27.73 | 25.18 | 28.44 | 20.72 | 24.49 | 21.2 | 28.13 | 25.45 | 35.68 |
| PKND | | | 39.01 | 30.67 | 22.41 | 43.85 | 26.3 | 29.36 | 24.14 | 28.29 |
| PKNE | | | | 28.09 | 20.73 | 44.8 | 26.3 | 29.69 | 25.33 | 32.25 |
| PKNF | | | | | 20.25 | 32.04 | 38.5 | 36.95 | 27.03 | 29.12 |
| PKNG | | | | | | 30.77 | 28.6 | 21.12 | 20.78 | 22.28 |
| PKNH | | | | | | | 26.6 | 29.61 | 23.06 | 29.14 |
| PKNI | | | | | | | | 30.06 | 21.9 | 25.54 |
| PKNJ | | | | | | | | | 25.37 | 27.93 |
| PKNK | | | | | | | | | | 28.61 |
| PKNL | | | | | | | | | | |

From the above homology matrix (Table 3) it is seen that the genes pknG and pknK are least homologous with other STPKs. However the general homology among the STPKs being low it was decided to look at how the genes cluster among each other when the clustering is done by near neighbour tree and average tree algorithm.

From the homology and clustering of the M. tuberculosis STPKs (FIG. 2 and FIG. 3) it seems that the genes pknG and pknK are outliers when compared to other STPKs.

STPKs in M. leprae

Compared to M. tuberculosis nearly half of the genes in M. leprae are either absent or pseudogenes. It is postulated that only the bare essential genes are present in M. leprae (Brosch R, Gordon S V, Eiglmeier K, Garnier T, Cole S T.

Comparative genomics of the leprosy and tubercle bacilli. Res. Microbiol 2000; 151:135-142). Of the 11 STPKs only four, pknA, pknB, pknG and pknL are present in M. leprae.

The above analysis strongly indicates that when all the factors, like homology to aceK, absence of transmembrane domain, outlier in STPKs, and presence in M. leprae are taken into account only pknG satisfies all the four characteristics. Thus we are satisified that pknG is the icd-kinase in M. tuberculosis.

### EXAMPLE 2

Phosphorylation of icd by pknG in M. tuberculosis

In order to demonstrate that pknG is the specific icd-kinase in M. tuberculosis we decided to show that it specifically phosphorylates the isocitrate dehydrogenase of Mycobacterium tuberculosis. Additionally this phosphorylation also inactivates the enzymic activity of icd1. To this end we have cloned, expressed and purified pknG and icd1.

Cloning and Purification of pknG:

The gene encoding pknG (Rv 0410c) was PCR amplified and cloned into the expression vector pET 21D. The recombinant plasmid was then transformed into the host BL21(DE3). Following induction by 1 mM IPTG for 2 hrs the cells were collected by centrifugation and lysed by sonication in Tris buffer (pH 7.5). Following sonication the cell lysate was centrifuged at 30000×g. It was seen that the protein was expressed in the soluble supernatant. Following this the protein was precipitated in 35% ammonium sulphate. The protein pellet was then dissolved in Tris buffer and purified through anion exchange (Q-Sepharose) chromatography. It was seen (FIG. 4) that pknG eluted around 300 nM NaCl. The purity of the protein was checked in 10% SDS-PAGE.

Cloning and Purification of Isocitrate Dehydrogenase:

There are two gene sequences (Rv 0066c and Rv3339c) annotated as isocitrate dehydrogenase in M. tuberculosis. However none of them have been proven experimentally to be the said enzyme. The smaller of the two genes icd1 (Rv 3339c) whose size is comparable to the E. coli gene was PCR amplified and cloned into the expression vector pET8c. The recombinant plasmid was then transformed into the host BL21(DE3). Following induction by 1 mM IPTG for 2 hrs the cells were collected by centrifugation and lysed by sonication in Tris buffer (pH 7.5). Following sonication the cell lysate was centrifuged at 30000×g. It was seen that the protein was expressed as inclusion bodies. The inclusion bodies were then purified in step (15 &60%) sucrose gradient. The purified inclusion bodies were then solubilized in 6M GnHCl and refolded by rapid dilution in PBS in presence of 1M NDSB. The purified protein was then monitored by 10% SDS-PAGE shown below

Specific Phosphorylation of icd1 by pknG

To prove that pknG specifically phosphorylates icd1 we set up a kinase reaction in a buffer (Tris-pH 7.4 containing 5 mM MgCl2, 2 mM MnCl2, 0.1 mM DTT and 0.1 mM Na Orthovanadate) the transfer molecule ATP was 0.1 mM in presence of γ32P ATP. The amount of proteins, pknG and icd1, in the reaction was 1 μg and 2.5 μg respectively. As a negative control E. coli icd protein was also used in a separate reaction. Following the kinase reaction the products were run on a SDS-PAGE and autoradiographed. The results are shown in FIG. 6.

The results clearly indicate that pknG specifically phosphorylates icd1 (lane 1) whereas when E. coli icd is added as a substrate, only autophosphorylation of pknG occurs (lane 2).

Inactivation of icd1 Due to Phosphorylation by pknG

So far we have shown that pknG specifically phosphorylated icd1 of M. tuberculosis. However in order for the branch point substrate diversion to occur it has to be shown that phosphorylation of icd1 causes its inactivation. In an earlier experiment it was shown that in pknG it is the serine residue that is phosphorylated (Koul et. al Microbiology 2001, vol147,2307-2314) hence it would be logical to assume that phosphorylation of icd1 by pknG occurs at the serine residue. The reaction catalysed by icd1 in the absence is be given by:
Isocitrate+NADP→α-ketogluterate+NADPH+2CO₂
By measuring the formation of NADPH at 340 nm we can monitor this reaction. However if the phosphorylation of icd1 by pknG causes the inactivation of the former then the formation of NADPH is reduced thereby reducing the absorbance at 340 nm. Shown below in FIG. 7 is the typical reaction of icd1 in presence and absence of pknG. It is obvious from the above result that phosphorylation of icd1 by pknG results in a concomitant inactivation of the former.
A Km Driven Shunt in Mycobacterium tuberculosis

There are two important features of the glyoxylate bypass enzymes which enables it to be operative in a micro organism. The first is the phosphorylation dependent inactivation of the icd1. This, as we have shown above by the specific phosphorylation of icd1 by pknG to be specifically operative in M.tb. The second is the differential Km for the substrate isocitrate between icd1 and icl. When the organism grows in glucose the preference of isocitrate for icd1 is governed by its differential Km. In order to verify if this is also true in M.tb we measured the Km of isocitrate for icd1. The Km as evaluated by Lineweaver-Burke plot and Cornish-Bowden direct linear plot indicated a value of 12 μM for isocitrate. The Km for icls have been previously determined by Russel and co-workers to be around 115 and 1500 μM for icl1 and icl2 respectively. This data clearly indicate that icd1 has a greater affinity to isocitrate over icls. This data proves the presence of a differential Km and we can thus conclude the possibility of a km driven shunt to be operative in M. tuberculosis.

In summary we disclose a novel method of pathway inactivation by substrate diversion. This concept is exemplified in M. tuberculosis by identification of a novel isocitrate dehydrogenase kinase (pknG) which specifically phosphorylates and inactivates its cognate isocitrate dehydrogenase (icd1). Thus inhibitors of pknG (compounds that bind such that the transfer of phosphate moiety to the corresponding serine residue in icd1 is prevented), will divert the substrate isocitrate away from the glyoxylate shunt. This will prevent the growth of the bacteria in C2 carbon source, which might be catastrophic when the bacteria are in the latent/persistence phase. It should be noted that pknG transfers the phosphoserine to icd1 by two-step procedure, in the first step the kinase is autophophorylated and in the second step the phosphate is transferred. There may be two types of inhibitors, the first that prevents autophosphorylation, the second that prevents the transfer. We believe that both the type of inhibitors will prevent the phosphorylation of icd1.

1. A method for attenuating a microorganism which comprises inhibiting in the microorganism a metabolic pathway essential for viability, by promoting use of the substrate of the pathway in a different metabolic pathway which is non-essential to the microorganism whereby the substrate is unavailable to the essential pathway and the micro organism is attenuated.

2. A method as claimed in claim 2 and wherein the presence or absence of a nutrient determines whether a metabolic pathway is essential or non-essential to the microorganism.

3. A method as claimed in claim 1 and wherein use of the substrate in the non-essential pathway is promoted by inhibition of an enzyme not in the essential pathway.

4. A method as claimed in claim 4 and wherein the enzyme to be inhibited is a kinase.

5. A method as claimed in claim 4 and wherein the enzyme to be inhibited is isocitrate dehydrogenase (icd1).

6. A method as claimed in claim 1 and wherein the microorganism is Mycobacterium tuberculosis.

7. A method as claimed in claim 6 and wherein the enzyme to be inhibited is pknG.

8. A method for identifying compounds that attenuate Mycobacterium tuberculosis which method comprises testing compounds in a test system for their ability to bind to pknG and prevent autophosphorylation.

9. A method for identifying compounds that attenuate Mycobacterium tuberculosis which method comprises testing compounds in a test system for their ability to bind to pknG and prevent phosphorylation of icd1 or a peptide derived from icd1.

10. A method for identifying compounds that attenuate Mycobacterium tuberculosis which method comprises testing compounds in a test system for their ability to bind to icd1 or a peptide derived from icd1 and prevent phosphorylation of icd1 by pknG.

11. A method for identifying compounds that attenuate Mycobacterium tuberculosis which method comprises testing compounds in a test system for their ability to prevent the phosphorylation and or inactivation of icd1.

12. An anti-microbacterial compound identified according to the method of any one of claims 8-11.
